(19) United States
(12) Patent Application Publication
(10) Pub. No.: US 2014/0199347 A1
(43) Pub. Date: Jul. 17, 2014

(54) ADJUVANT COMPOSITIONS WITH 4-1BBL

(71) Applicant: Haval Shirwan, Louisville, KY (US)

(72) Inventor: Haval Shirwan, Louisville, KY (US)

(73) Assignee: UNIVERSITY OF LOUISVILLE RESEARCH FOUNDATION, INC.

(21) Appl. No.: 13/983,940

(22) PCT Filed: Feb. 7, 2012
(86) PCT No.: PCT/US12/24092

§ 371 Date: Mar. 13, 2014

Related U.S. Application Data
(60) Provisional application No. 61/441,392, filed on Feb. 10, 2011.

(51) Int. Cl. A61K 39/00; A61K 31/739; A61K 38/19

(52) U.S. Cl. CPC A61K 39/0011; A61K 38/191; A61K 31/739
USPC 424/277.1; 514/21.2; 424/184.1

(57) ABSTRACT

Streptavidin(SA)-4-1BBL and TLR agonists such as monophosphoryl lipid A (MPL) exhibit surprising synergy as adjuvants, inducing immune responses against weak antigens. Accordingly, there are provided adjuvant compositions comprising 4-1BBL and a toll-like receptor (TLR) agonist, such as MPL, methods of inducing an immune response against an antigen in a subject, comprising administering to the subject (a) the antigen, (b) a TLR agonist and, (c) 4-1BBL, and methods of treating a tumor or a cancer in a subject, comprising administering to the subject (a) an antigen associated with the tumor or cancer, (b) a TLR agonist, and (c) 4-1BBL.

### CROSS REFERENCE TO RELATED APPLICATIONS

This application claims benefit of the filing date of U.S. Provisional Application No. 61/441,392, filed Feb. 10, 2011.

### U.S. GOVERNMENT FUNDING

This work was funded in part by grants from the NIH, Kentucky Lung Cancer Research Program, W. M. Keck Foundation, and the Commonwealth of Kentucky Research Challenge Trust Fund. This invention was made with government support under Grants R41 CA121665, R44 AI071618, and R43AI074176 awarded by the National Institutes of Health. The government has certain rights in the invention

### FIELD OF THE INVENTION

The present invention generally relates to the surprisingly effective synergy observed when combining a toll-like receptor (TLR) agonist such as monophosphoryl lipid A (MPL) with 4-1BBL as adjuvants, and to related compositions and methods to enhance the immune response against antigens, and related methods of preventing and treating diseases, including cancer.

### BACKGROUND

Therapeutic vaccines are preferred alternatives to conventional treatments for cancer primarily because of their safety profile and generation of long-term immunological memory that is critical for the control of disease recurrence, which is the main cause of death from cancer. Therapeutic vaccines based on tumor associated antigens (TAAs) are particularly attractive because of their ease of production, scale-up, storage, and administration to a broad patient population. The efficacy of such vaccines, however, is curtailed by the weak antigenic nature of self TAAs due to both central and peripheral tolerogenic mechanisms (1, 2). Despite continued promising results, there is a greater than two-decade track record of failure with cancer vaccines. Similar failures are also found in other immunity contexts, including vaccines against infectious diseases, self antigens, and against haptens such as toxins and addictive drugs.

3-O-desacyl-4′-monophosphoryl lipid A (MPL), is a nontoxic version of lipopolysaccharide approved by the FDA to be used as the adjuvant component of a preventive vaccine against HPV infection (5). MPL is also under investigation as an adjuvant in other contexts. MPL is a toll-like receptor 4 (TLR-4) agonist, like lipid A, lipopolysaccharide and other structurally related compounds. CpG is a structurally dissimilar TLR agonist. MPL primarily targets innate immunity, leading to the recruitment, activation, and maturation of antigen presenting cells (APCs), such as dendritic cells (DCs) that facilitate the generation of adaptive immune responses (6).

4-1BBL is a member of the TNF family. 4-1BBL is a costimulatory molecule that targets CD8⁺ T cells for activation, acquisition of effector function, survival, and long-term memory (15-17). 4-1BBL is also able to stimulate CD4⁺ Treg cells to induce tolerance against a given antigen (U.S. Pat. No. 7,745,215). 4-1BBL is expressed on the cell surface and has no function in soluble form. The extracellular functional domain of 4-1BBL can be fused to a modified form of streptavidin (SA) to generate a chimeric molecule (SA-4-1BBL) that exists as tetramers and oligomers owing to the structural features of SA (12).

### SUMMARY OF THE INVENTION

The present invention is drawn to the surprising discovery of the synergy of toll-like receptor (TLR) agonists such as monophosphoryl lipid A (MPL) and 4-1BBL as an adjuvant composition to induce immune responses against a co-administered antigen. In some embodiments there are provided compositions comprising 4-1BBL and a TLR agonist. In some embodiments, the TLR agonist is a TLR4 agonist, such as lipopolysaccharide, lipid A or a chemical analogue, such as MPL. In some embodiments, there are provided compositions comprising 4-1BBL and MPL. 4-1BBL may be provided in a fusion protein, such as a streptavidin-4-1BBL fusion protein. In some embodiments, the 4-1BBL fusion protein forms higher order structures, such as trimers and aggregates, via the streptavidin moieties. In some embodiments, the composition further comprises a pharmaceutically-acceptable excipient.

In further embodiments, the composition further comprises an antigen. The antigen may be provided in a conjugate with 4-1BBL, such as a conjugate comprising biotinylated antigen complexed with SA-4-1BBL. Suitable antigens include a tumor or cancer-associated antigen, a self antigen, an antigen associated with an infectious agent, or a hapten. Suitable cancer or tumor-associate antigen include: human telomerase reverse transcriptase (hTERT), survivin, MAGE-1, MAGE-3, human chorionic gonadotropin, carcinoembryonic antigen, alpha fetoprotein, pancreatic oncofetal antigen, MUC-1, CA 125, CA 15-3, CA 19-9, CA 549, CA 195, prostate-specific antigens, prostate-specific membrane antigen, Her2/neu, gp-100, mutant K-ras proteins, mutant p53, truncated epidermal growth factor receptor, chimeric protein ^{p210}BCR-ABL, E7 protein of human papilloma virus, EBNA3 protein of Epstein-Barr virus, cTAGE-1 and variants, BLA or globotriaosylceramide (P^{k} antigen), human T-cell leukemia virus-associated cell membrane antigens (HTLV-MA), Thymocyte surface antigen JL1, Adult T cell leukemia associated, human retrovirus associated antigen (ATLA), Anaplastic lymphoma kinase (ALK), fusion proteins (NPM/ALK and variants), Common acute lymphoblastic leukemia antigen (CALLA), Immunoglobulin Id, Type II glycoproteins (such as HM1.24, KW-2, KW-4, KW-5, KW-12), Oncofetal antigen immature laminin receptor protein (OFA-iLRP), and EBV proteins (e.g., LMP2A).

Antigens associated with an infectious agent include those from HIV, influenza, malaria, tuberculosis, staphylococcus, and streptococcus.

Also provided are methods of inducing an immune response against an antigen in a subject, comprising administering to the subject (a) the antigen, (b) a TLR agonist, and (c) 4-1BBL, such as SA-4-1BBL. Suitable TLR agonists include TLR4 agonists, such as lipopolysaccharide, lipid A and chemical analogues, such as MPL. A second and/or subsequent administrations may also be performed. Again, the antigen may be provided in a conjugate with 4-1BBL. In practicing the methods, any two or more of the antigen, TLR agonist (such as MPL), and 4-1BBL may be administered as part of the same composition, or may be administered simultaneously or sequentially in separate compositions by the same or different routes of administration.

Also provided are methods of treating a tumor or a cancer in a subject, comprising administering to the subject (a) an antigen associated with the tumor or cancer, (b) a TLR agonist, such as MPL, and (c) 4-1BBL, such as SA-4-1BBL. Suitable TLR agonists include TLR4 agonists, such as lipopolysaccharide, lipid A and chemical analogues, such as MPL. A second and/or subsequent administrations may also be performed with the same or different antigen. Again, the antigen may be provided in a conjugate with 4-1BBL. Again, any two or more of the antigen, TLR agonist (such as MPL), and 4-1BBL may be administered as part of the same composition, or may be administered simultaneously or sequentially in separate compositions by the same or different routes of administration.

In any of the embodiments described herein, a further immune costimulatory molecule, such as OX-40L/CD137L, can be used in addition to the 4-1BBL The further immune costimulatory molecule (e.g., OX-40L/CD 137L) can be provided in a fusion protein comprising avidin or streptavidin. In practicing the methods, the further immune costimulatory molecule may be administered as part of the same composition as any one or more of the antigen, TLR agonist (such as MPL), and 4-1BBL, or may be administered simultaneously or sequentially in separate compositions by the same or different routes of administration.

### BRIEF DESCRIPTION OF THE DRAWINGS

FIG. 1. A single vaccination with the SA-4-1BBL/MPL adjuvant system results in the eradication of established TC-1 tumor in mice. (A) C57BL/6 mice were challenged subcutaneously with 1×10⁵ live TC-1 cells and left unvaccinated (PBS) or vaccinated once subcutaneously on day 6 post-tumor challenge with E7 (50 μg) mixed with control SA protein (10 μg) or SA-4-1BBL (25 μg), MPL (25 μg), or the combination of both agents (25 μg/agent). The log-rank test and Kaplan-Meier method were used for analyses. *P<0.05 as compared to all the other groups, but SA-4-1BBL that was not significant (ns). (B) Data from (A) are presented for individual animals in each group.

FIG. 2. Vaccination with the SA-4-1BBL/MPL adjuvant system induces strong anti-tumor CD8⁺ T cell effector and memory responses that correlate with vaccine efficacy. Long-term (>90 days) surviving mice were boosted with the same indicated vaccine formulations used for primary vaccination. Lymph node cells were harvested 7 days later and assessed for E7₄₉₋₅₇ peptide-specific CD8⁺ T cells expressing (A) intracellular IFN-γ mono, (B) IFN-γTNF-α double, and (C) IFN-γTNF-αIL-2 triple cytokines. (D) Splenocytes from the same groups were phenotyped to test the percentage of effector memory CD44^{hi}CD62L^{low}CD8⁺ T cells. Data for each panel are representative of two independent experiments that include 3-4 mice per group. P values were as shown and calculated using one way ANOVA and Tukey HSD test (ns=not significant).

FIG. 3. Vaccination with the SA-4-1BBL/MPL adjuvant system results in an increase in the intratumoral Teff/Treg cell ratio. Mice bearing TC-1 tumor (˜3-4 mm in diameter; n=4 per group) were vaccinated subcutaneously with E7 protein (50 μg) alone or with SA-4-1BBL (25 MPL (25 μg), or a combination of both agents (25 μg/agent). One week post-vaccination, tumors were harvested and stained for intratumoral CD8⁺ T cells and CD4⁺Foxp3⁺ Treg cells followed by analysis using confocal microscopy. (A) Confocal pictures of tumor sections showing CD4⁺Foxp3⁺ Treg cells (top panel; bright cells) stained with anti-CD4 antibody, anti-Foxp3 antibody, and Hoechst, and CD8⁺ T cells (bottom panel; bright spots) stained with anti-CD8 antibody and Hoechst. (B) Quantitative analysis of intratumoral CD4⁺Foxp3⁺ Treg cells, (C) CD8⁺ T cells, and (D) CD8⁺ Teff/Treg cell ratio. P values were as shown and calculated using one way ANOVA and Tukey HSD test (ns=not significant).

FIG. 4. Therapeutic efficacy of SA-4-1BBL/MPL adjuvant system requires CD8⁺ T cells while Treg cells compromise the efficacy of MPL monotherapy. CD8⁺ T cells and Treg cells were depleted using antibodies against CD8 and CD4 molecules, respectively, one day before vaccination with E7 TAA and the indicated adjuvant system using the TC-1 established tumor model. Data for PBS, E7+MPL, and E7+MPL+SA-4-1BBL groups were taken from FIG. 1.

FIG. 5. Vaccination with the SA-4-1BBL/MPL adjuvant system generates potent therapeutic response in the 3LL lung metastasis model. Mice (n=4-5/group) were challenged with 2×10⁵ live 3LL cells by intravenous tail injection and vaccinated once subcutaneously on day 6 or twice on days 6 and 13 post-tumor challenge with survivin (SVN) (50 μg) alone or antigen with SA-4-1BBL (25 MPL (25 μg), or a combination of both agents (25 μg/agent). (A) Lungs were harvested 27 days post tumor challenge and assessed for tumor growth by weight and macroscopic presence of tumor nodules. (B) Intracellular IFN-γ response of CD8⁺ T cells was assessed after with phorbol 12-myristate 13-acetate (PMA) and ionomycin stimulation of lymphocytes harvested from mice in (A). (C) Lungs harvested from mice with two vaccinations are assessed as in (A). P values were as shown and calculated using one-way ANOVA and Post Hoc LSD test (ns=not significant).

FIG. 6. Vaccination with the SA-4-1BBL/MPL adjuvant system does not promote autoimmunity. Sera were harvested from mice challenged with 3LL tumor cells shown in FIG. 5A and TC-1 tumor cells in FIG. 1A at the experimental end points and tested for the presence of autoantibodies against single stranded DNA (ssDNA) in ELISA. Serum pooled from a minimum of 3 naïve and 3 lupus mice were used as negative and positive controls, respectively.

FIG. 7. Vaccination with the SA-4-1BBL/MPL adjuvant system induces robust primary CD8⁺ T cell effector functions. (A) Tumor draining lymphocytes from mice (n=4) shown in FIG. 3 were stimulated with E7₄₉₋₅₇ peptide and analyzed for CD8⁺ T cell intracellular IFN-γ expression. (B) Splenocytes from mice in (A) were stimulated with E7₄₉₋₅₇ peptide and IL-2 for 5 days and used as effectors against TC-1 tumors. 3LL tumor cells were used as irrelevant targets. The data is representative of 3 independent experiments. P values were as shown and calculated using one-way ANOVA and Tukey HSD test (ns=not significant).

FIG. 8 depicts the amino acid sequences of representative fusion proteins, (A) core streptavidin (CSA) with the extracellular domain of murine 4-1BBL and (B) core streptavidin (CSA) with the extracellular domain of human 4-1BBL. The core streptavidin sequence is underlined. (C) Fusion of hexahistidine-streptavidin-ALA3 tag onto murine 4-1BBL (6XHis-SA-ALA3-m4-1BBL). The 6XHis-SA-ALA3 tag is underlined. (D) Fusion of Flag streptavidin and Ala3 tags onto human CD137 ligand (1XFlag-SA-Ala3-CD137L). The 1XFlag-SA-Ala3 tag is underlined.

### DETAILED DESCRIPTION OF THE INVENTION

For the purposes of the present application, the following terms have these definitions:

As used herein “a” or “an” means one or more, unless specifically indicated to mean only one.

“Administration” as used herein encompasses all suitable means of providing a substance to a patient. Common routes include oral, sublingual, transmucosal, transdermal, rectal, vaginal, subcutaneous, intramuscular, intravenous, intra-arterial, intrathecal, via catheter, via implant etc. In some embodiments, a composition is administered near or directly to the tumor, such as by direct injection into the tumor or injection into the blood such as when the tumor is a tumor of the blood.

An “adjuvant” increases the immune response against an antigen with which it is presented. Adjuvants are known in the art and include aluminium hydroxide, aluminium phosphate, monophosphoryl lipid A, oils, cytokines, and the like. Oil-in-water and water-in-oil emulsions also are used as adjuvants. Antigen carriers such as virosomes and immune-stimulating complexes (e.g., ISCOM and ISCOMATRIX) also may result in enhanced presentation of antigens. (See Table 1 of Leroux-Roels, Vaccine 28S: C25-C36 (2010))

“Antigen” is used herein without limitation. Antigens include proteins, lipids, sugars, nucleic acids, chemical moieties, and other moieties that induce an immune response. Antigens include proteins, which may or may not be modified, such as by glycosylation or methylation, that are cyclized or bound to lipids, for example. Antigens associated with an infectious agent or disease include antigens that are part of the infectious agent, such as envelope proteins, capsid proteins, surface proteins, toxins, cell walls, antigenic lipids, and the like. Other antigens may be expressed only in the presence of the host. Other suitable antigens may, in some embodiments, include antigens of the host, including those that are induced, modified or otherwise overexpressed as a marker of infection or disease. All such antigens that are derived from, or associated with, an infectious agent, an infection, a condition or disease, are suitable for use in the present invention. Also suitable for use as an “antigen” in accordance with the present invention are peptides comprising antigenic portions of full-length proteins, such as peptides comprising a portion of a protein that induces an immune response, such as an immunogenic epitope. For example, suitable antigens may include synthetic peptides that induce an immune response.

In some embodiments, the antigen is an “allergen.” An allergen is an antigen which induces the symptoms of allergic disease, such as IgE antibodies against the allergen, MAST cell degranulation and/or the release of histamine in the presence of the allergen. In such an embodiment, the goal may be less concerned with developing an immune response to the antigen, per se, but rather in changing the nature of the immune response. For example, inducing an immune response to the allergen that stimulates the production of IgG, instead of IgE antibodies. This may be achieved through the induction of a T_{H1} over T_{H2} response.

“Excipients” includes vehicles, carriers, diluents, pH adjusting and/or buffering agents, tonicity adjusting agents, stabilizers, wetting agents, binders, and the like. Pharmaceutically acceptable excipients are well known in the art. For example, alum (hydrated potassium aluminium sulfate, KA1 (SO₄)₂.12H₂O), is commonly used in vaccine formulations as an excipient to bind and stabilize biological molecules and as an adjuvant.

“Immune co-stimulatory polypeptide” means a polypeptide molecule that increases an individual's immune response against a pathogen (including an infectious agent) or tumor. In addition to 4-1BBL, OX40L is an exemplary immune co-stimulatory polypeptide that can be used in the compositions and methods described herein.

“Immune cell” as used herein includes any cell that is involved in the generation, regulation or effect of the acquired or innate immune system. Immune cells include T cells such as CD4+ cells, CD8+ cells and various other T cell subsets, B cells, natural killer cells, macrophages, monocytes, dendritic cells, and neutrophils.

“Patient” or “subject” as used herein includes any mammal. In some embodiments, the patient is human. A person of ordinary skill in the art will recognize that particular immune co-stimulatory molecules, signaling molecules, cell markers, cell types, infectious agents etc., discussed with reference to one species, may have corresponding analogues in different species, and that such analogues, and their use in corresponding and related species, are encompassed by the present invention.

“Toll-like receptor agonist” (or TLR) as used herein are molecules which bind to and activate toll-like receptors. Agonists and antagonists of different TLRs are known. Lipid A is a lipopolysaccharide found in the outer membrane of gram negative bacteria that acts as a potent TLR4 agonist. MPL is a chemical analogue of lipid A that also acts as a TLR4 agonist.

“Tumor” as used herein includes solid and non solid tumors (such as leukemia); and different stages of tumor development from pre-cancerous lesions and benign tumors, to cancerous, malignant and metastatic tumors.

A “vaccine” describes a preparation designed to induce an immune response against an antigen. A vaccine may be therapeutic, given during treatment to boost the immune response or drive the response in a specific direction, or it may be prophylactic or preventative, given prior to or shortly after exposure to a disease. A vaccine does not have to induce a fully protective response that prevents or eradicates all evidence of disease, as not all vaccines produce an immune response in all people, and the strength and nature of the immune response varies between people. A vaccine may be both therapeutic and prophylactic at the same time, in treating an existing condition and preventing future recurrences.

### Toll-Like Receptor Agonists.

The toll-like receptors are a family of proteins involved in innate immunity.

Toll-like receptors and agonists

| | | | Origin of the |
|---|---|---|---|
| TLR | Localization | Ligand | Ligand |
| TLR1 | Plasma membrane | Triacyl | Bacteria |
| | | lipoprotein | |
| TLR2 | Plasma membrane | Lipoprotein | Bacteria, viruses, |
| | | | parasites, self |
| TLR3 | Endolysosome | dsRNA | Virus |
| TLR4 | Plasma membrane | LPS | Bacteria, viruses, |
| | | | self |
| TLR5 | Plasma membrane | Flagellin | Bacteria |
| TLR6 | Plasma membrane | Diacyl lipoprotein | |
| | | Bacteria, viruses | |
| TLR7 | Endolysosome | ssRNA | Virus, bacteria, |
| (human TLR8) | | | self |
| TLR9 | Endolysosome | CpG-DNA | Virus, bacteria, |
| | | | protozoa, self |
| TLR10 | Endolysosome | Unknown | Unknown |
| TLR11 | Plasma membrane | Profilin-like | Protozoa |
| | | molecule | |

### (See Table 1 of Takeuchi & Akira, Cell 140: 805-820 (2010)).

TLR agonists in clinical use or development include
(a) MPL, a TLR4 agonist and a chemical analogue of lipidA/LPS. MPL may also be used in combination with alum or QS21 (a saponin);
(b) synthetic derivatives of dsRNA (TLR3);
(c) S. typhimurium flagellin (TLR5);
(d) imiquidazoquinoline derivatives (TLR7 and/or 8); and
(e) Immunostimulatory sequences such as synthetic phosphorothioate-linked DNA oligonucleotides with optimized CpG motifs (TLR9)
See Table 1 of Leroux-Roels, Vaccine 28S: C25-C36 (2010); See also Table 1 of Coffman et al., Immunity 33: 492-503 (2010).

### 4-1BBL

Immune co-stimulatory molecules are involved in the natural interaction between naïve T cells and antigen presenting cells, which results in their reciprocal activation and prompts the expression of various cell surface ligands and receptors, and soluble proteins that contribute to the initiation, maintenance, and long-term memory of the immune response. At least three signals are required for the initial activation of naïve T cells. Signal 1 is generated by interactions between a T cell receptor (TCR) and a nominal peptide presented by major histocompatibility complex (MHC) molecules on the surface of professional APC, such as dendritic cells (DC). Signal 2 can be mediated by several different molecules and is important to a sustained immune response. Signal 3 is transduced via cytokines elaborated by activated T cells and APC and is important to the maintenance of effector immune responses.

4-1BBL (also known as 4-BB-L, 4-BB ligand, TNFSF9, ILA ligand) is a type II protein expressed on activated B cells, macrophages, and DC two to three days following activation. 4-1BB/4-1BBL interactions also transduce Signal 2 to CD8⁺ T cells in a CD28-independent manner and stimulate them to produce cytokines, expand, and acquire effector functions.

4-1BBL contains 254 amino acids (26624 Da). See Alderson et al. Eur J Immunol. 1994 September; 24(9):2219-27. The full amino acid sequence of human 4-1BBL can be found under accession no. P41273 in the Swiss-Prot database. 4-1BBL is a type II glycoprotein with residues 1-28 forming a potential cytoplasmic domain, residues 29-49 forming a single predicted transmembrane domain, residues 50-254 forming a potential extraceulluar domain, and residues 35-41 representing a poly-Leu stretch. The nucleotide sequence in humans encoding the 4-1BBL can be found in GenBank accession no. NM_{—}003811. Residues 50-254 of 4-1BBL or fragments thereof that can bind to its cognate receptor 4-1BB, can be linked or expressed as a fusion with a binding pair member for use in accordance with the present invention. U.S. Pat. No. 7,598,345.

Unless specified herein as “full-length,” reference herein to 4-1BBL polypeptide encompasses the full-length polypeptide as well as fragments or portions thereof that exhibit immune co-stimulatory function, including, but not limited to those fragments and portions specifically identified below. Thus, for example, reference to a 4-1 BBL polypeptide connotes a polypeptide comprising a fragment or portion of full-length 4-1 BBL that exhibits immune co-stimulatory function, such as the extracellular domain of 4-1 BBL or the full-length 4-1BBL protein. In some embodiments, the immune co-stimulatory polypeptide does not comprise the transmembrane domain of 4-1BBL. In some embodiments, the immune co-stimulatory polypeptide comprises at least the extracellular domain of 4-1 BBL, or a receptor-binding fragment thereof. “IMMUNOSTIMULATORY COMPOSITIONS AND METHODS” describes suitable fusion proteins between 4-1BBL and streptavidin.

### Additional Immune Costimulatory Polypeptides

In any of the embodiments described herein, a further immune costimulatory molecule, can be included in the composition in addition to the 4-1BBL. The further immune costimulatory molecule can be provided in a fusion protein comprising avidin or streptavidin.

Immune co-stimulatory polypeptides include, without limitation, LIGHT, CD80 (B7-1), CD86 (B7-2), ICOS, ICOSL (including B7h, B7-H2, B7RP-1, GL-50 and LICOS), CD94 (KP43), CD40L (CD154), ICAM-1 (CD54), ICAM-2, ICAM-3, SLAM (CD150), HAS (CD24), 4-1BB (CDw137), OX40L, CD28, CD40 (BP50), CD25 (IL-2Rα), Lymphotoxin (LTα or LTβ), TNF, Fas-L, GITR (activation-inducible TNRF), GITR Ligand, CD11a (α_{L} integrin), CD11b (α_{M} integrin), L-selectin (CD62L), CD69 (very early activation antigen), CD70 (CD27L), PD-L1, PD-L2, B7-H3, B7-H4, OX40L, 4-1BBL, CD27L, CD30L, LIGHT, BAFF, and APRIL. See, e.g., Watts & DeBenedette, 1999, Curr. Opin. Immunol., 11:286-93.

In some embodiments, the compositions and methods of the invention further comprise OX40L. OX40L is expressed by dendritic cells and other APC and binds to OX40 which is present on activated T cells. OX40L contains 183 amino acids (21950 Da). See Miura et al. Mol. Cell. Biol. 11:1313-1325 (1991). The full amino acid sequence of OX40L can be found under accession no. P23510 in the Swiss-Prot database. OX40L is a type II glycoprotein with a cytoplasmic domain at residues 1-23, a transmembrane domain at residues 24-50 and an extracellular domain at residues 51-183. The nucleotide sequence of OX40L is 3510 bp, with the coding sequence being 157-708 (see Genbank accession no. NM 003326.2). Residues 51-183, or fragments thereof of OX40L that can bind to its cognate receptor OX40, can be linked or expressed as a C-terminal fusion to a binding pair member for use in accordance with the present invention. U.S. Pat. No. 7,598,345 for “IMMUNOSTIMULATORY COMPOSITIONS AND METHODS” describes suitable fusion proteins between OX40L and streptavidin.

### Fusion Proteins

In exemplary embodiments, the 4-1BBL is comprised in a fusion protein with streptavidin (SA) or avidin, or fragments thereof which retain substantial properties of the full-length protein. Such fragments include “core streptavidin” (“CSA”), a truncated version of the full-length streptavidin polypeptide which may include streptavidin residues 13-138, 14-138, 13-139 or 14-139. The nucleic acid sequences encoding streptavidin and avidin and the streptavidin and avidin amino acid sequences can be found, for example, in GenBank Accession Nos. X65082; X03591; NM_{—}205320; X05343; 221611; and Z21554. When another immune costimulatory peptide is used, such as OX-40L, it also may be provided in a fusion protein with SA or avidin.

SA and CSA are able to aggregate into trimers and higher order structures; thus, SA-4-1BBL conjugates (or other immune costimulatory conjugates, such as SA-OX-40L conjugates) may form trimers and higher order structures that may be necessary for immune costimulation. Another property of SA, CSA and avidin is binding to biotin, and fragments with at least 50% or more of the binding affinity of native SA or avidin, respectively, also may be used. The biotin-binding property may be used to target or localize 4-1BBL (or other immune costimulatory molecule, such as OX-40L) to a target site or surface, or to attach another molecule, such as an antigen.

### Antigens & Infectious Agents

The methods and compositions described herein are useful for generating or enhancing an immune response against any antigen or infectious agent, including TAAs, antigens associated with an infectious agent, and an infectious agent itself. An antigen associated with the targeted tumor or infectious agent (or the infectious agent itself) may be presented to immune cells, thereby generating or enhancing an immune response.

### 1. TAAs

In one embodiment, the antigen is a tumor associated antigen (TAA), and the compositions and methods provide cancer immunotherapy effective to generate or enhance a patient's immune response against a tumor. In accordance with this embodiment, the methods and compositions may reduce tumor size and/or inhibit the growth of tumor cells.

Representative tumor cells which may be targeted include, without limitation, carcinomas, which may be derived from any of various body organs including lung, liver, breast, bladder, stomach, colon, pancreas, skin, and the like. Carcinomas may include adenocarcinoma, which develop in an organ or gland, and squamous cell carcinoma, which originate in the squamous epithelium. Other cancers that can be treated include sarcomas, such as osteosarcoma or osteogenic sarcoma (bone), chondrosarcoma (cartilage), leiomyosarcoma (smooth muscle), rhabdomyosarcoma (skeletal muscle), mesothelial sarcoma or mesothelioma (membranous lining of body cavities), fibrosarcoma (fibrous tissue), angiosarcoma or hemangioendothelioma (blood vessels), liposarcoma (adipose tissue), glioma or astrocytoma (neurogenic connective tissue found in the brain), myxosarcoma (primitive embryonic connective tissue), an esenchymous or mixed mesodermal tumor (mixed connective tissue types). In addition myelomas, leukemias, and lymphomas are also susceptible to treatment.

A number of TAAs associated with specific tumor types have been identified. These include human telomerase reverse transcriptase (hTERT), survivin, MAGE-1, MAGE-3, human chorionic gonadotropin, carcinoembryonic antigen, alpha fetoprotein, pancreatic oncofetal antigen, MUC-1, CA 125, CA 15-3, CA 19-9, CA 549, CA 195, prostate-specific antigens; prostate-specific membrane antigen, Her2/neu, gp-100, mutant K-ras proteins, mutant p53, truncated epidermal growth factor receptor, chimeric protein ^{p210}BCR-ABL; E7 protein of human papilloma virus, and EBNA3 protein of Epstein-Barr virus. Any of these antigens, antigenic fragments thereof, and mixtures of antigens and/or fragments can be used in accordance with the compositions and methods described herein to generate or enhance a patient's anti-tumor immune response. Table 5 lists some exemplary TAAs and diseases associated with such TAAs.

TABLE 5

| Antigen | Diseases |
|---|---|
| cTAGE-1 and variants | Cutaneous T cell lymphoma |
| BLA or globotriaosylceramide | Burkitt's lymhoma |
| (P^{k} antigen) | |
| human T-cell leukemia virus-associated | Adult T-cell leukemia |
| cell membrane antigens (HTLV-MA) | lymphoma (ATL) |
| Thymocyte surface antigen JL1 | Majority of acute leukemias |
| Adult T cell leukemia associated, human | Adult T cell leukemia |
| retrovirus associated antigen (ATLA) | |
| Epstein-Barr virus (EPV) antigens | Burkitt's lymphoma, |
| | Hodgkin's disease |
| Anaplastic lymphoma kinase (ALK), | CD30+ anaplastic large cell |
| fusion proteins (NPM/ALK and variants) | lymphoma (ALCL) |
| Common acute lymphoblastic leukemia | Most acute lymphoblastic |
| antigen (CALLA) | leukemias |
| Immunoglobulin Id; Type II | Lymphoproliferative |
| glycoproteins (e.g., HM1.24; KW-2, | diseases |
| KW-4, KW-5, KW-12); Oncofetal | |
| antigen immature laminin receptor | |
| protein (OFA-iLRP); EBV proteins | |
| (e.g., LMP2A) | |

Additional human TAAs recognized by T-cells may be found in, for example, Novellino et al. “A listing of human tumor antigens recognized by T cells: March 2004 update” Cancer Immunology and Immunotherapy, 54: 187-207 (2005) which is incorporated by reference herein. Many animal TAAs corresponding to animal corollaries of these diseases, and to other animal diseases, are known in the art and also included within the scope of the invention.

In one embodiment, the TAA is selected from the group consisting of human telomerase reverse transcriptase (hTERT) and survivin. hTERT is expressed in >85% of human cancers, while its expression is restricted in normal tissues. See, e.g., Vonderheide et al., Immunity 1999, 10: 673-79. Similarly, survivin, which has been identified as an inhibitor of apoptosis, is absent from normal tissues but expressed in most tumor types including lung, colon, pancreas, prostate and breast cancer. See, e.g., Ambrosini et al., Nat. Med. 1997, 3: 917-21. Because these TAAs are expressed in the majority of cancer types and are rare or absent from normal tissues, they are attractive antigens for use in cancer immunotherapy methods according to the present invention.

In another embodiment, the TAA is associated with cervical cancer. The viral transforming proteins, E6 and E7 (also known as “early” proteins), are consistently expressed in cervical cancer cell lines and in HPV-associated cancers, and are consistently expressed in most cervical cancers. Because E6 and E7 are completely foreign viral proteins, and may harbor more antigenic peptides or epitopes than a mutant protein, and thus have several benefits as a TAA for therapeutic purposes.

TAAs may be admixed with 4-1BBL and MPL for administration. The TAA may also be conjugated to, for example, 4-1BBL, such as through a biotin linkage.

### 2. Infectious Agents

Representative infectious agents against which the compositions and methods described herein may be applied include, without limitation, any virus, bacteria, fungi or protozoan. Table 6 lists examples of infectious agents.

TABLE 6

| | ETIOLOGICAL | | ASSOCIATED |
|---|---|---|---|
| | AGENT | GENUS | DISEASE |
| BACTERIAL | Mycobacterium | | Tuberculosis |
| | tuberculosis | | |
| | Bacillus anthracis | | Anthrax |
| | Staphylococcus | | Sepsis |
| | aureus | | |
| | Borrelia spp | | Lyme disease |
| | Salmonella | | Typhoid |
| VIRAL | Adenoviridae | Mastadenovirus | Infectious canine |
| | | | hepatitis |
| | Arenaviridae | Arenavirus | Lymphocytic |
| | | | choriomeningitis |
| | Caliciviridae | Norovirus | Norwalk virus |
| | | | infection |
| | Coronaviridae | Coronavirus | Severe Acute |
| | | | Respiratory Syndrome |
| | | Torovirus | |
| | Enterovirus | | Hand-foot and mouth |
| | | | disease, diarrheal |
| | | | disease |
| | Filoviridae | Marburgvirus | Viral hemorrhagic |
| | | | fevers |
| | | Ebolavirus | Viral hemorrhagic |
| | | | fevers |
| | Flaviviridae | Flavivirus | West Nile Encephalitis |
| | | Hepacivirus | Hepatitis C virus |
| | | | infection |
| | | Pestivirus | Bovine Virus Diarrhea, |
| | | | Classical swine fever |
| | Hepadnaviridae | Orthohepadnavirus | Hepatitis |
| | Herpesviridae | Simplexvirus | cold sores, genital |
| | | | herpes, bovine |
| | | | mammillitis |
| | | Varicellovirus | chickenpox, shingles, |
| | | | abortion in horses, |
| | | | encephalitis in cattle |
| | | Cytomegalovirus | infectious |
| | | | mononucleosis |
| | | Mardivirus | Marek's disease |
| | Orthomyxoviridae | Influenzavirus A | Influenza |
| | | Influenzavirus B | Influenza |
| | Papillomaviridae | Papillomavirus | Skin warts (including |
| | | | genital warts), skin |
| | | | cancer, cervical cancer |
| | Picornaviridae | Enterovirus | Polio |
| | | Rhinovirus | Common cold |
| | | Aphthovirus | Foot-and-mouth disease |
| | | Hepatovirus | Hepatitis |
| | Poxviridae | Orthopoxvirus | Cowpox, vaccinia, |
| | | | smallpox |
| | Reoviridae | Rotaviruses | Diarrhea |
| | | Orbivirus | Blue tongue disease |
| | Retroviridae | Gammaretrovirus | Feline leukemia |
| | | Deltaretrovirus | Bovine leukemia |
| | | Lentivirus | Human |
| | | | immunodeficiency, FIV, |
| | | | and SIV |
| | Rhabdoviridae | Lyssavirus | Rabies |
| | | Ephemerovirus | Bovine ephemeral fever |
| | Togaviridae | Alphavirus | Eastern and Western |
| | | | equine encephalitis |
| PARASITIC | | Plasmodium | Malaria |
| | | Leishmania | Leishmaniasis |
| FUNGAL | | Aspergillis | |
| | | Candida | |
| | | Coccidia | |
| | | Cryptococci | |
| | | Geotricha | |
| | | Histoplasma | |
| | | Microsporidia | |
| | | Pneumocystis | |

Human and avian influenza, HIV, hepatitis C, tuberculosis, West Nile Virus, cryptococcosis (meningitis) herpes, chlamydia, and anthrax are representative of infectious agents. Any antigen associated with the infectious agent can be used in accordance with the invention. Any infectious agent may be used, such as a virus, including a human or avian influenza virus or HIV, or any other virus. Other infectious agents and antigens derived therefrom include Hepatitis A, C or E virus, Japanese encephalitis virus, Dengue virus, Hantavirus, Rabies virus, and SARS coronavirus. The infectious agent may be modified or attenuated to reduce or eliminate its infectivity.

In accordance with one embodiment, the antigen or infectious agent is provided in a conjugate with 4-1BBL, such as by biotinylation of the agent and binding to the SA moiety on SA-4-1BBL (e.g., forming an Antigen-Biotin-SA-4-1BBL complex).

For the purpose of illustration only, this aspect is described in more detail with reference to influenza. Influenza is a contagious disease caused by the influenza virus, and affects the respiratory tract, often resulting in symptoms in the nose, throat and lungs, as well as fever, headache, tiredness and aches. It can also lead to complications such as pneumonia, bronchitis, or sinus and ear infections or exacerbate chronic conditions. Influenza viruses are classified as type A, B or C. Strains belonging to types A and B circulate in the population and are associated with most cases of human influenza. Type A influenza causes the overwhelming majority of public health problems in humans. In this context, the antigen may comprise one or more of H1 and N1 (both highly immunogenic) and/or one or more of nucleoprotein (NP) and matrix protein 1 (MP1) and/or matrix protein 2 (MP2) (all highly conserved, structural proteins). Proteins from pandemic strains such as H5, also can be used as antigens in accordance with the invention.

The compositions and methods described herein can be used in influenza vaccines that are easy to produce and manufacture quickly, whose antigenic component can be changed and updated based on the current health needs without difficulty, that selectively targets viral machinery and infected cells, and that can be administered post-infection for a therapeutic effect as well as pre-infection for prevention.

The compositions and methods described herein are useful as vaccines against other infectious agents, which can be selected in an analogous manner by those skilled in the art, based on the antigens associated with those infectious agents. For example, antigens associated with HIV include HIV envelope gp120 epitopes (e.g., variable loops such as V3), or other HIV proteins such as Gag proteins (Pr55^{gag}, matrix p17, capsid p24, nucleocapsid p7), p5; Pol (polymerase), Vif (viral infectivity factor p23); Vpr (viral protein R p15); Rev (regulator of viral gene expression p19); Vpu (viral protein U); Env (gp 160, gp120, 41); Tat (transcriptional activator p14); and Nef (negative effector p24). See, e.g., Peters, 201, Vaccine 2: 688-705; Michael, 2003, Clin. Med. 3: 269-72; Gandhi & Walker, 2002, Ann. Rev. Med. 53: 149-72; Haseltine, 1991, FASEB 5: 2349-60. Other antigens useful in vaccines include capsular polysaccharides of Haemophilus influenzae type b, capsular polysaccharides of Neisseria meningitidis, capsular polysaccharides of Streptococcus pneumoniae, surface antigens of Hepatitis B, and inactivated exotoxins of diphtheria and tetanus toxins. These antigens can be used in accordance with the composition and methods described above with reference to influenza antigens.

### Allergens

The term “allergen” refers to antigens associated with allergies. An allergic response is characterized by the release of inflammatory factors, particularly histamine, leading to pathologic inflammation in an individual. Allergies are, typically, also associated with IgE antibodies directed against the allergens. Examples of allergens include, but are not limited to: pollens (e.g. grass, ragweed, birch and mountain cedar); house dust and dust mites; mammalian epidermal allergens and animal danders; mold and fungus; insect bodies and insect venom; feathers; food; and drugs (e.g., penicillin). The compositions and methods of the invention are suitable to induce or modulate an immune response against an allergen that is of a different nature to a preexisting immune response. For example, an individual possessing a T_{H2} immune response against an allergen such that IgE antibodies are produced upon exposure to the allergen may be induced, by embodiments of the present invention, to produce a T_{H1} immune response against the allergen, that counteracts the allergy inducing T_{H2} response and so alleviate allergic disease.

### Compositions

As set forth above, some embodiments of the invention relate to compositions. These include compositions comprising 4-1BBL and a toll-like receptor (TLR) agonist, such as MPL. 4-1BBL may be provided in a fusion protein, such as a streptavidin-4-1BBL fusion protein or a core streptavidin-4-1-BBL fusion protein. The compositions may also include pharmaceutically-acceptable excipients that are well-known in the art, and may be prepared and/or provided in suitable forms for storage (frozen, lyophilized, etc.) and/or administration. Some excipients may also exhibit adjuvant properties. An example of such an excipient is alum (hydrated potassium aluminium sulfate (Potassium alum) with the formula KAl(SO₄)₂.12H₂O). Alum can bind to and stabilize variant antigens, and is also used as an adjuvant. The 4-1BBL/TLR agonist composition is suitable for use as an adjuvant, and therefore may include or be administered with an antigen. The antigen may be included in the 4-1BBL/TLR agonist composition at the time of manufacture, or can be added later, prior to or upon administration. The antigen may be a separate component of the composition, or may be part of a conjugate that also comprises 4-1BBL, such as may be prepared by biotinylation of the antigen and binding to the streptavidin moiety of SA-4-1BBL. As discussed above, any antigen can be used, such as from cancer, tumors, and infectious diseases.

In any of the embodiments described herein, a further immune costimulatory molecule, such as OX-40L/CD137L, can be included in the composition in addition to the 4-1BBL The further immune costimulatory molecule (e.g., OX-40L/CD137L) can be provided in a fusion protein comprising avidin or streptavidin.

### Methods

As set forth above, some embodiments of the invention relate to methods. For example, the methods described herein are useful to induce an immune response against an antigen, such as an antigen associated with tumors or cancers, or infectious agents. In some embodiments, such methods comprise administering to a subject (a) the antigen, (b) a toll-like receptor (TLR) agonist, such as MPL, and (c) 4-1BBL. The methods described herein also are useful for treating a tumor or cancer by administering to a subject (a) the antigen, (b) a toll-like receptor (TLR) agonist, such as MPL, and (c) 4-1BBL. In any of these methods, a second and/or subsequent administrations of a same or similar agents (e.g., the same or different antigen) may be performed. As noted above with reference to compositions, the 4-1BBL may be provided in a fusion protein, such as a streptavidin-4-1BBL fusion protein or a core streptavidin-4-1-BBL fusion protein and/or the antigen may be provided as a separate component or in a conjugate that also comprises 4-1BBL. Further, in practicing the methods, any two or more of the antigen, TLR agonist, and 4-1BBL may be administered as part of the same composition, or may be administered simultaneously or sequentially in separate compositions by the same or different routes of administration.

In any of the embodiments described herein, a further immune costimulatory molecule, such as OX-40L/CD137L, can be administered in addition to the 4-1BBL The further immune costimulatory molecule (e.g., OX-40L/CD137L) can be provided in a fusion protein comprising avidin or streptavidin, and can be provided in the same composition as the 4-1BBL or can be provided in a separate composition that is administered simultaneously with or sequentially (i.e., before or after) the 4-1BBL composition.

Thus, in practicing the methods described herein, one may administer antigen, a TLR agonist, 4-1BBL, and optional additional costimulatory molecules, simultaneously in the same or different compositions, or sequentially, at the same or different sites, and at the same or different times and in different orders.

The following examples illustrate the invention in more detail, and are not intended to limit the scope of the invention in any respect.

### EXAMPLES

### Example 1

### 4-1BBL/MPL Synergy

### Materials and Methods

### Mice and Cell Lines

C57BL/6 and C57BL/6.SJL mice were bred in a barrier animal facility at the University of Louisville. All animals were cared for in accordance with institutional and NIH guidelines. TC-1 and 3LL cell lines were purchased from ATCC (Manassas, Va.) and maintained as published (7).

### Antibodies and Other Reagents

Fluorochrome-conjugated anti-CD8-APC-Cy7, anti-CD62L-PE, anti-CD44-APC, anti-TNF-PE, anti-IFN-γ-PE-Cy7, and anti-IL-2-PerCp-Cy5.5, and isotype controls were purchased from BD Bioscience, eBioscience, and BioLegend. MPL was purchased from InvivoGen (San Diego, Calif.). The HPV16 RAHYNIVTF E7 peptide (E7₄₉₋₅₇), SA-4-1BBL, E7 and mouse SVN proteins were reported previously (7).

### Tumor Models and Vaccination

C57BL/6 mice were challenged subcutaneously with 1×10⁵ live TC-1 cells into the right flank. For therapy, mice were vaccinated subcutaneously on day 6 post-tumor challenge with various vaccine formulations containing E7 protein (50 μg) alone as control or with SA-4-1BBL (25 MPL (25 μg), or the combination of both agents (25 μg/agent). The doses of E7, SA-4-1BBL, and MPL used in this study were based on previously published studies (7). Mice were euthanized when tumor reached a size of 12 mm in diameter, ulcerated, or mice showed signs of discomfort. CD8⁺ and CD4⁺ T cells were depleted using antibodies against CD8 (clone 53.6.72) and CD4 (clone GK 1.5) at 500 μg/mice via intra-peritoneal injection one day before vaccination

For the pulmonary tumor model, 2×10⁵ live 3LL cells were injected intravenously into the tail vein of mice. Mice were vaccinated subcutaneously either once on day 6 or twice on days 6 and 13 post-tumor challenge with various vaccine formulations containing survivin (SVN) protein (50 μg) alone as control or with SA-4-1BBL (25 μg), MPL (25 gig), or the combination of both agents (25 μg/agent). Mice were euthanized 27 days post-tumor challenge for analysis of lung tumor burden as described (12, 18).

### Flow Cytometry and Confocal Microscopy

Spleens and/or tumor draining lymph nodes (TdLNs) were processed as described previously (7). For memory T cell typing, lymphocytes were stained with anti-CD8-APC-Cy7, anti-CD62L-FITC, and anti-CD44-APC antibodies. For intracellular cytokine staining, lymphocytes (1×10⁶ cells/mL) were stimulated either with 10 μg/mL E7₄₉₋₅₇ peptide for 2 hrs followed by incubation with GolgiPlug (1 μl/mL, BD PharMingen) overnight or with phorbol 12-myristate 13-acetate (PMA) (5 ng/ml, Sigma) and ionomycin (500 ng/ml, Sigma) for 2 hrs followed by incubation with GolgiPlug (1 μl/ml) for an additional 4 hrs. Cells were first stained with anti-CD44-APC and anti-CD8-APC-Cy7, fixed with 4% paraformaldehyde, and then stained with anti-IFN-γ-PE-Cy7, anti-IL-2-Percp-Cy5.5, anti-TNF-PE, or isotype controls followed by acquisition and analysis as previously reported (12). Intratumoral CD8⁺ T cells and CD4⁺Foxp3⁺ Treg cells were analyzed using confocal microscopy as described (12).

Analysis of Autoantibody to ssDNA

A single stranded DNA (ssDNA) ELISA was performed to assess the presence of auto-antibodies in treated mice as described (19). Briefly, ninety six titer plates coated with 1 μg/well of heat-denatured calf thymus DNA (ssDNA, Sigma) were blocked with PBS containing 5% BSA+0.5% Tween 20+0.1% naïve C57BL/6 serum. Serum dilutions were added to wells and incubated at 4° C. overnight. Wells were washed 3 times, incubated with anti-mouse IgG-HRP, and absorbance was measured at 450 nm.

### Cytotoxicity Assay

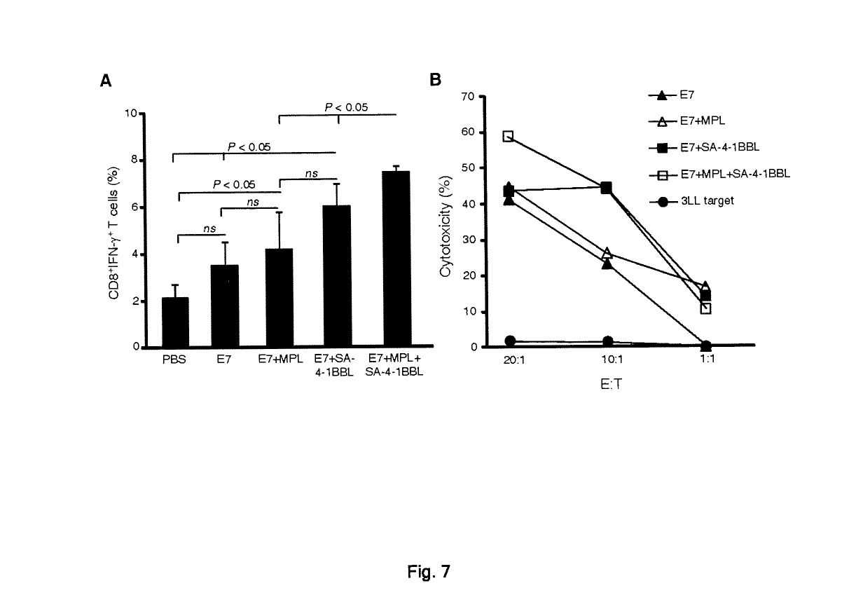

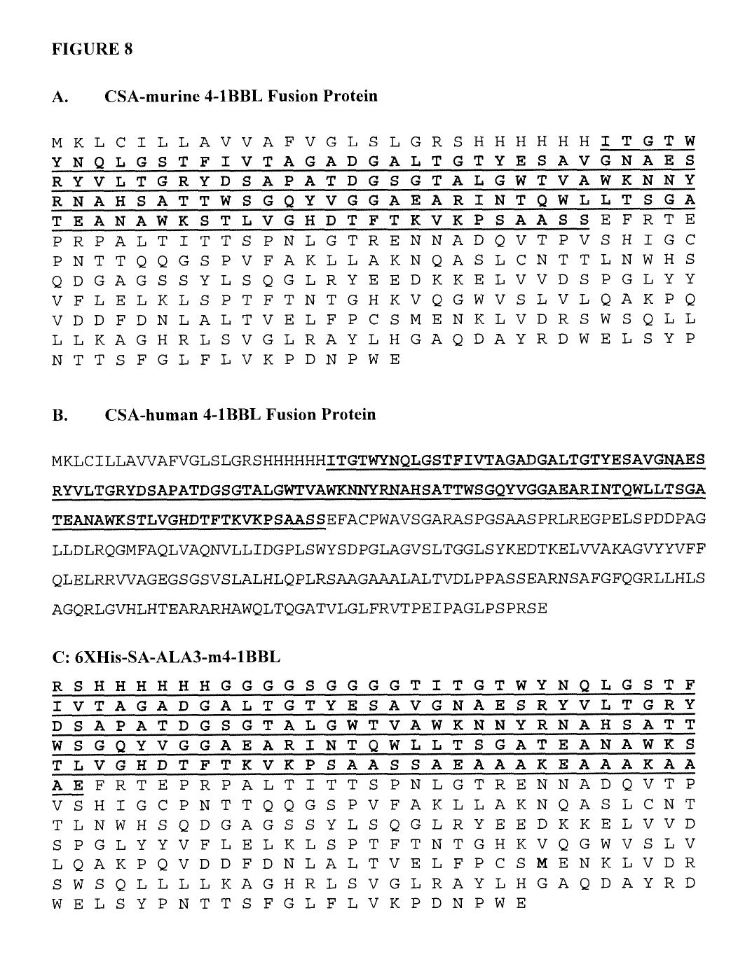

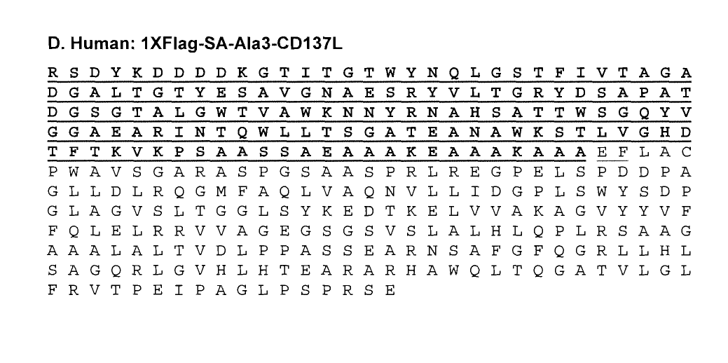

Mice with ˜3-4 mm TC-1 tumors in diameter were vaccinated subcutaneously with vaccine formulations containing E7 protein (50 μg) alone or with SA-4-1BBL (25 μg), MPL (25 μg), or the combination of both agents (25 μg/agent). One week post-vaccination, splenocytes were cultured with 10 μg of E7₄₉₋₅₇ peptide/mL in complete MLR medium supplemented with 50 IU/mL of IL-2. Viable lymphocytes were harvested 5 days later using a Ficoll gradient and used as effectors at various ratios against TC-1 target cells for 4 hrs as described (41). See FIGS. 7A and 7B.

### Results

### Combined Use of SA-4-1BBL and MPL as the Adjuvant Component of E7 TAA-Based Vaccine has Robust Efficacy in Eradicating Established TC-1 Tumors

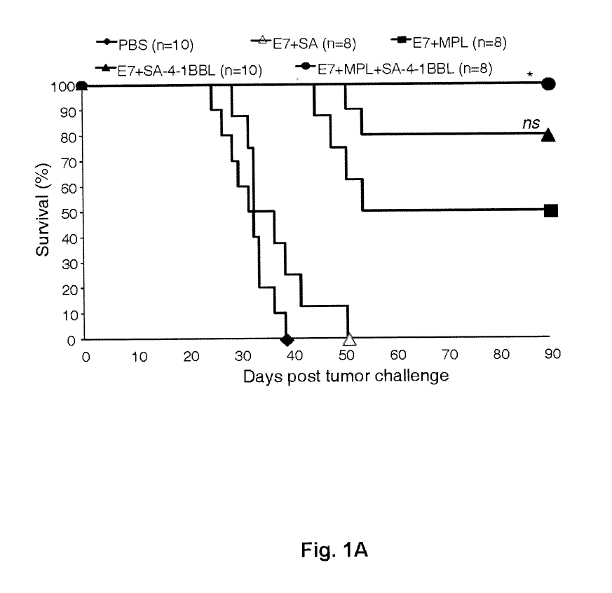

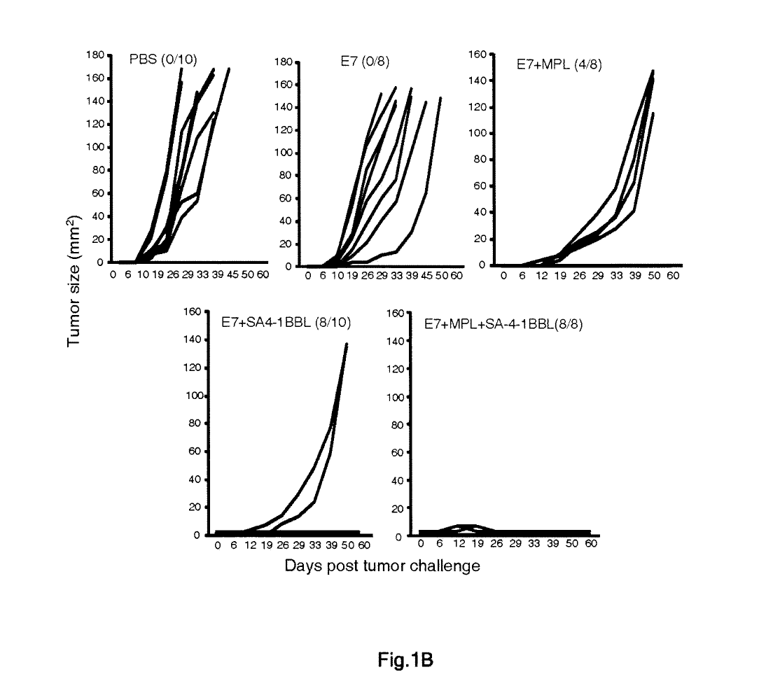

A single vaccination with SA-4-1BBL and E7 protein was effective in eradicating E7 expressing established TC-1 tumors in >70% of mice (12). Although impressive, the present inventors have discovered that the therapeutic efficacy of this vaccine can further be improved by modifying the formulation to include MPL as a second adjuvant with a primary effect on innate immunity. A single subcutaneous vaccination with E7 protein mixed with SA-4-1BBL and MPL resulted in the eradication of established TC-1 tumors in all mice, which remained tumor-free over an observation period of 90 days (FIG. 1A). It was completely surprising to achieve 100% effectiveness, i.e. in all mice, and for such an extended period.

By comparison, monotherapy with SA-4-1BBL or MPL resulted in eradication of tumors in only 80% and 50% of mice, respectively. Mice that expired from tumors in the single agent groups, however, had slow kinetics of tumor progression as compared with both PBS and E7 protein control groups, where all mice expired from the tumor burden within 50 days (FIG. 1B). Taken together, these data demonstrate that SA-4-1BBL/MPL as an adjuvant system is effective in eradicating the established TC-1 tumors with better therapeutic efficacy than the individual agents, and that SA-4-1BBL has better efficacy than MPL.

The Therapeutic Efficacy of the Vaccine is Associated with the Synergistic Effects of SA-4-1BBL and MPL on the Generation of Peripheral CD8⁺ T Cell Responses

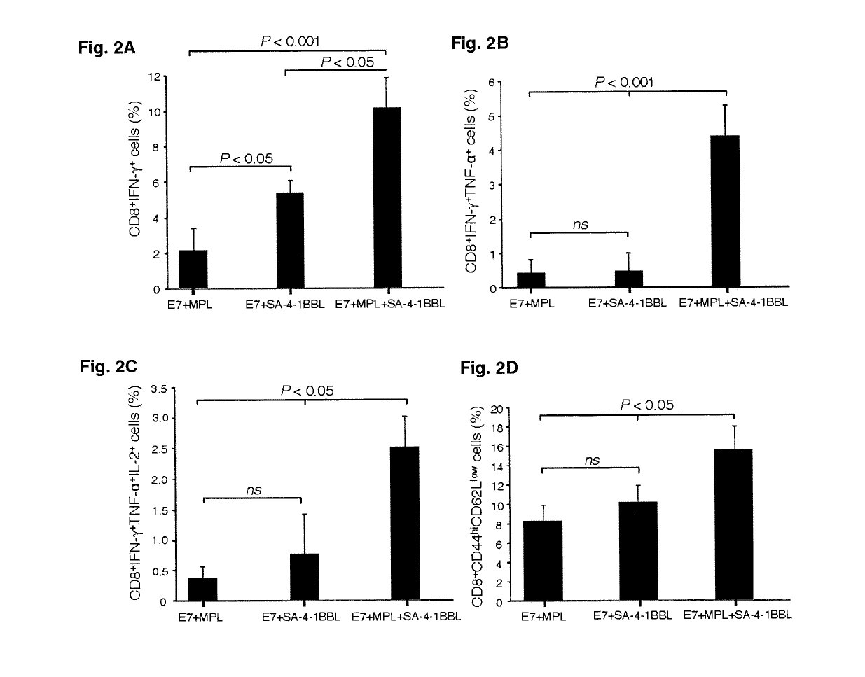

CD8⁺ T cell effector and memory responses are critical to the elimination of the primary tumor and control of recurrences, respectively, in various tumor settings, including the TC-1 model (7, 10, 11, 13). The CD8⁺ T cell effector and long-term memory responses elicited by various vaccine formulations were assessed as follows. Mice that had eradicated the tumor in response to various vaccine formulations were boosted subcutaneously with the same formulations and then euthanized one week later to test the intracellular cytokine response of CD8⁺ T cells to the dominant E7₄₉₋₅₇ epitope (10). Consistent with the therapeutic efficacy, vaccination with E7 protein and SA-4-1 BBL/MPL generated a better antigen-specific cytokine response than single adjuvant therapy as assessed by CD8⁺ T cell expressing IL-2, IFN-γ, and TNF-α triple cytokines (FIG. 2A-C). Consistent with the therapeutic responses, mice vaccinated with SA-4-1BBL formulation generated significantly (P<0.05) better IFN-γ response than the MPL formulation (FIG. 2A). A vaccine formulation with SA-4-1BBL/MPL also generated the most effective CD8⁺ T cell memory recall responses as compared to those including SA-4-1BBL and MPL as single adjuvants (FIG. 2D). Collectively, these data demonstrate that SA-4-1BBL and MPL adjuvants work in synergy to generate potent CD8⁺ T cell effector and memory responses that correlate with the therapeutic efficacy of the vaccine against the TC-1 tumor.

Vaccination with the SA-4-1BBL/MPL Adjuvant System Results in a Favorable Intratumoral CD8⁺ Teff/Treg Cell Ratio

Elevated levels of intratumoral CD4⁺Foxp3⁺ Treg cells along with a decline in CD8⁺ Teff cells is associated with a clinically unfavorable prognosis of cancer patients (21, 22) and depletion of Treg cells results in better immune efficacy of therapeutic vaccines (23, 24). The effect of the SA-4-1BBL/MPL adjuvant system on the status of intratumoral Treg and Teff cells was evaluated as follows:

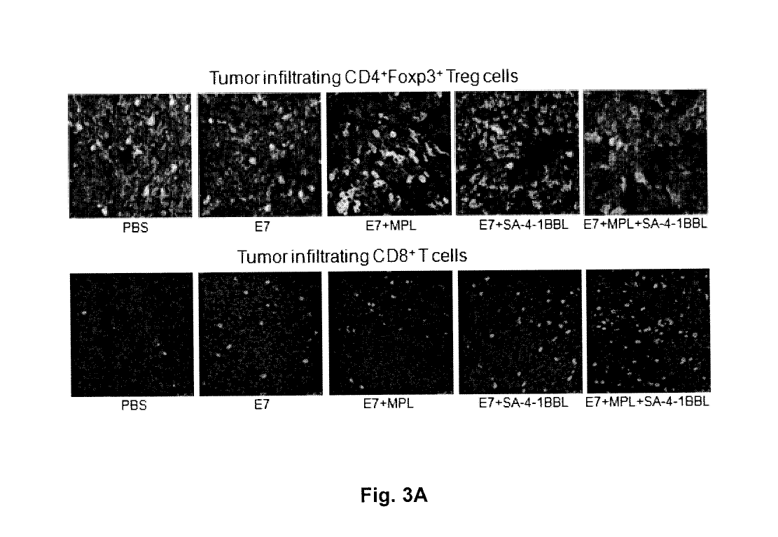

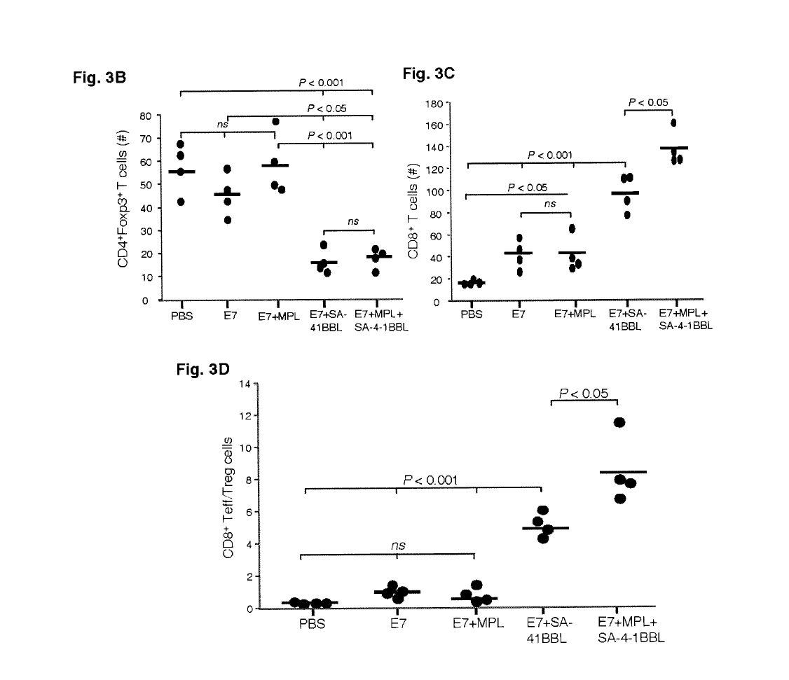

Mice bearing ˜3-4 mm TC-1 tumor were vaccinated subcutaneously with various vaccine formulations. One week post-vaccination, tumors were harvested and analyzed for the presence of intratumoral CD8⁺ T cells and CD4⁺FoxP3⁺ Treg cells using confocal microscopy. There was a significant reduction in the number of intratumoral Treg cells in mice vaccinated with either SA-4-1BBL as a single adjuvant or in combination with MPL when compared with PBS controls or E7 protein alone. (FIG. 3A, B). Interestingly, vaccine formulation containing MPL as a single adjuvant did not have detectable effect on the number of intratumoral Treg cells as compared with PBS control, and indeed performed worse than E7 protein alone that appreciably, but not statistically significantly, reduced the intratumoral number of Treg cells.

The following data show that a decrease in the number of Treg cells caused by SA-4-1BBL/MPL or SA-4-1BBL as monotherapy inversely correlates with the number of intratumoral CD8⁺ T cells, a hallmark of a successful immunotherapeutic approach against cancer (25). Vaccination with SA-4-1BBL/MPL had the most pronounced effect on the number of intratumoral CD8⁺ T cells infiltration, followed by SA-4-1BBL, whereas MPL had a moderate effect that was similar to the E7 protein alone (FIG. 3C). This increased intratumoral CD8⁺ T cells by SA-4-1BBL/MPL resulted into the most favorable intratumoral Teff/Treg cell ratio, followed by SA-4-1BBL as monotherapy (FIG. 3D). In marked contrast, MPL as a single adjuvant had no effect on the intratumoral Teff/Treg cell ratio as compared with both PBS and E7 protein controls. Significantly (P<0.05) better E7 TAA-specific CD8⁺ T cell IFN-γ (FIG. 7A) as well as TC-1 killing (FIG. 7B) responses were observed in mice vaccinated with both adjuvants as compared with a single adjuvant. Consistent with the therapeutic efficacy and infiltration of intratumoral CD8⁺ T cells, SA-4-1BBL as monotherapy generated better CD8⁺ T cell IFN-γ as well as killing responses than E7 antigen alone, whereas MPL failed to do so. Taken together, these findings demonstrate that SA-4-1BBL and MPL work in synergy to increase the intratumoral Teff/Treg cell ratio that correlates with the potent efficacy of this adjuvant system in eliminating established tumors.

CD8⁺ T Cells are Correlated with the Therapeutic Efficacy of SA-4-1BBL/MPL Adjuvant System while Treg Cells are Detrimental to the Efficacy of MPL Monotherapy

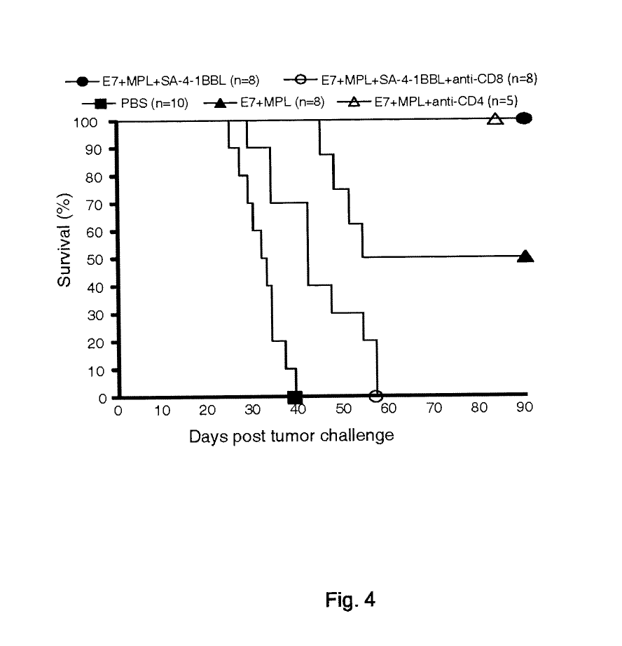

To test if a high CD8⁺ Teff/Treg cell ratio can serve as a predictor of vaccine therapeutic efficacy, we used antibodies against CD8 and CD4 molecules to deplete CD8⁺ Teff and Treg cells, respectively. Mice with established TC-1 tumors were treated with depleting antibodies one day before vaccination with E7 protein admixed with SA-4-1BBL/MPL or MPL as monotherapy. As shown in FIG. 4, depletion of CD8⁺ T cells completely abrogated the therapeutic efficacy of SA-4-1BBL/MPL adjuvant system, while depletion of CD4⁺ T cells, including Treg cells, improved the therapeutic efficacy of MPL from 50 to 100%. Taken together, these data provide direct evidence for the opposing roles of CD8⁺ T and Treg cells in vaccine efficacy and point to the importance of Teff/Treg cell ratio as a predictor of vaccine efficacy/failure.

Vaccination with SA-4-1BBL/MPL Adjuvant System and Survivin Eradicates Established 3LL Pulmonary Metastatic Tumors

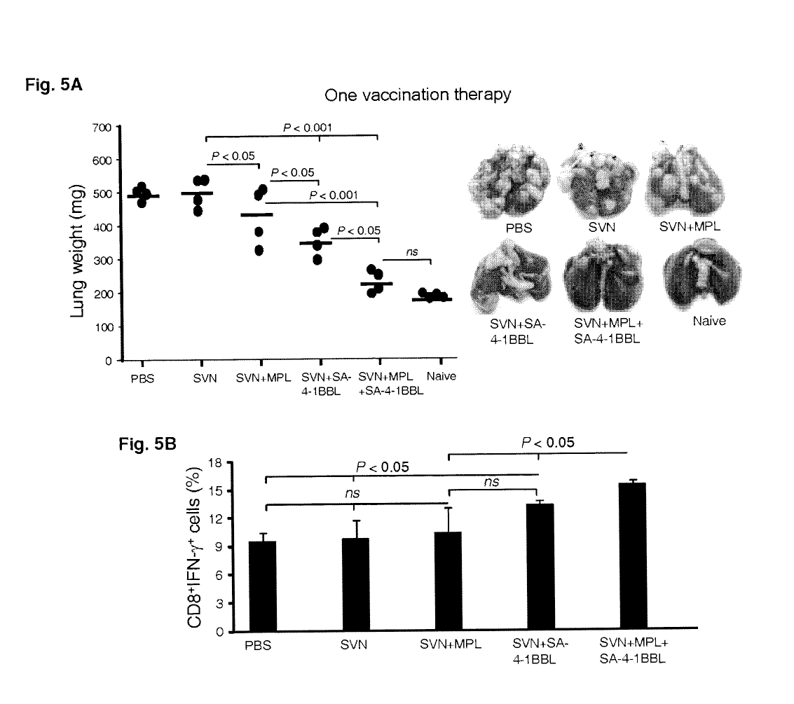

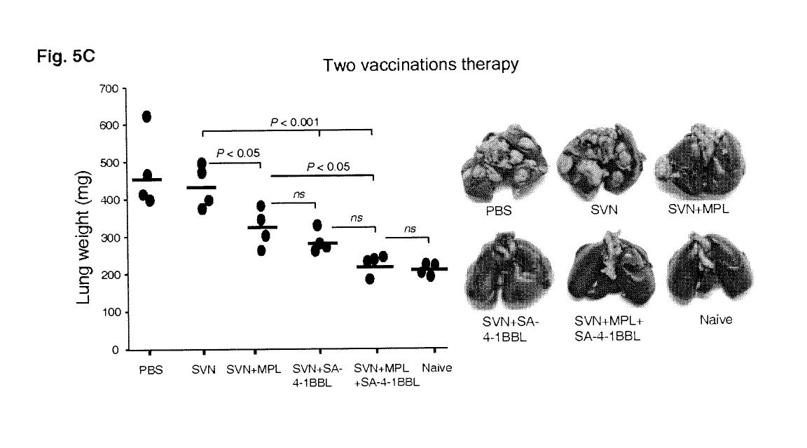

Efficacy of vaccination with survivin (SVN), a weak and potentially tolerant self-TAA, was assessed in the 3LL pulmonary metastasis model. Mice were challenged intravenously with a lethal dose of live 3LL cells followed by subcutaneous vaccination on day 6 with various formulations containing SVN recombinant protein and SA-4-1BBL and/or MPL as adjuvants. As shown in FIG. 5A, the vaccine formulation containing both adjuvants had the most therapeutic efficacy over the single adjuvant composition in controlling tumor growth, as demonstrated both by lung weight and presence of tumor nodules. Similar to the TC-1 model, the vaccine formulation containing SA-4-1BBL as sole adjuvant had better efficacy in controlling tumor growth than MPL as sole adjuvant, which had a statistically significant (P<0.05) effect in controlling tumor growth over the controls of PBS or adjuvant-free SVN. The therapeutic efficacy of SA-4-1BBL/MPL combination therapy, or SA-4-1BBL monotherapy, but not MPL monotherapy, correlated with significantly (P<0.05) higher number of CD8⁺ T cells expressing IFN-γ⁺ as compared with PBS and SVN alone controls (FIG. 5B).

Although lungs of SA-4-1BBL/MPL vaccinated mice had similar weights as compared with lungs of naïve mice, some of the lungs had microscopically detectable tumor nodules. We therefore, tested the efficacy of a booster injection 7 days after the first vaccination. As shown in FIG. 5C, boosting with SA-4-1BBL/MPL with SVN resulted in complete eradication of lung tumor in all mice. Booster vaccination with single adjuvants was also effective in eradicating and/or controlling tumor burden that reached statistical significance (P<0.05) as compared with PBS and SVN alone controls. Collectively, these findings further confirm the utility of SA-4-1BBL/MPL as a powerful adjuvant system to elicit potent immune responses to a self-TAA that translates into effective immunotherapy in a stringent pulmonary preclinical metastasis model.

### Therapeutic Efficacy of the SA-4-1BBL/MPL Adjuvant System is Achieved in the Absence of Detectable Clinical Toxicity and Autoimmunity

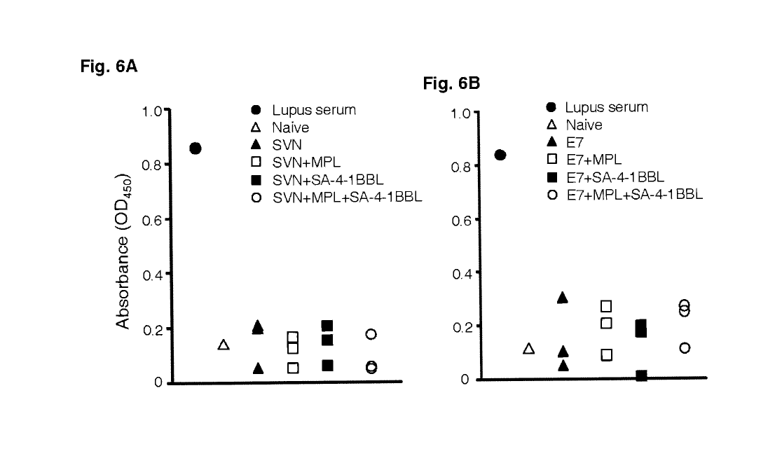

Autoimmunity is a potential setback to effective self-TAA-based therapeutic vaccine formulations using potent adjuvants to induce immune responses to such antigens (26). Given the potent therapeutic activity of the adjuvant system described herein, we tested serum from mice with successful immunotherapy for both the TC-1 as well as 3LL models, for the presence of antibodies against single stranded DNA (ssDNA) as a sign of systemic autoimmunity. There was lack of significant amount of auto-antibodies to ssDNA in all the groups tested, whereas the serum from mice with full blown lupus had high levels of such antibodies (FIG. 6). Importantly, signs of acute toxicity in vaccinated mice were not detected, including weight loss, unexpected mortality, gross anatomy, and macroscopic analysis of body organs, demonstrating the safety profile of this adjuvant system.

### Discussion

MPL and SA-4-1BBL as the adjuvant component of an HPV E7 TAA-based vaccine synergized to generate a robust primary CD8⁺ T cell effector and long-term memory responses that translated into improved therapeutic efficacy in the TC-1 cervical cancer mouse model. The therapeutic efficacy of the adjuvant system was totally dependent on CD8⁺ T cells and associated with a favorable intratumoral CD8⁺ Teff/CD4⁺Foxp3⁺ Treg cell ratio. Moreover, the synergy of the adjuvant system is not limited to the xenogenic E7 TAA. A vaccine formulation containing the adjuvant combination with SVN, as a bona fide self-TAA, was equally effective in eradicating/controlling tumors in the 3LL metastatic pulmonary cancer model.

While not wishing to be bound by theory, it would appear that MPL in the adjuvant system synergizes with SA-4-1BBL for the activation of CD8⁺ T cells through the activation of DCs and antigen cross-presentation (27), resulting in the upregulation of 4-1BB receptor on the surface of CD8⁺ T cells that in turn become the direct target of SA-4-1BBL. This scheme is supported by our data reported herein, showing that MPL synergizes with SA-4-1BBL in generating robust CD8⁺ T cell primary and long-term memory responses that translate into effective therapy in two different established tumor models, TC-1 cervical and 3LL pulmonary carcinoma, with two different antigens, HPV E7 xenogenic and SVN bona fide self-TAA antigens. Consistent with this hypothesis, depletion of CD8⁺ T cells one day before vaccination completely abrogated the efficacy of the SA-4-1BBL/MPL adjuvant system in eradicating TC-1 tumors. In addition to its direct effect on CD8⁺ T cells, SA-4-1BBL may also augment the effect of MPL on DCs by improving their antigen uptake and cross-presentation. This hypothesis is supported by observations that a subpopulation of DCs constitutively express 4-1BB receptor (28, 29), and vaccination with SA-4-1BBL enhances their antigen uptake and cross-presentation (7, 12).

CD4⁺CD25⁺FoxP3⁺ Treg cells play a critical role in immune evasion mechanisms employed by acute (30) as well as chronic infections (31, 32) and cancer (2, 23, 33), and as such serve as an important barrier for the efficacy of vaccines. Therefore, vaccine formulations that specifically control the number and/or function of Treg cells while enhancing the number of Teff cells may have desired therapeutic efficacy in settings of cancer and chronic infections. Consistent with this hypothesis are studies demonstrating that the physical depletion of Treg cells or modulation of their regulatory function using antibodies to various cell surface markers have protective and therapeutic effects against various tumors in preclinical models (33-37). A recent study using mice transgenically expressing the diphtheria toxin receptor only in Treg cells demonstrated that specific and conditional depletion of these cells protected mice from carcinogenesis-induced spontaneous tumors via innate immunity and eradicated established tumors via CD8⁺ T cell- and IFN-γ-dependent responses (36). Consistent with preclinical studies, Treg cells were shown to accumulate in various progressing cancers in patients and a high intratumoral Teff/Treg cells ratio is considered the hallmark of a favorable prognosis (21-23).

Important in this context are the results reported here, showing a robust increase in the ratio of intratumoral CD8⁺ Teff/Treg cells in response to vaccination with the SA-4-1BBL/MPL adjuvant system. Vaccination with SA-4-1BBL as monotherapy also significantly improved the intratumoral CD8⁺ Teff/Treg cell ratio. Surprisingly, MPL as monotherapy was not only inefficient in significantly increasing the frequency of intratumoral CD8⁺ T cell infiltration, but also failed to decrease the intratumoral number of Treg cells, resulting in an unfavorable CD8⁺ Teff/Treg cell ratio. The Treg cells played a detrimental role in the efficacy of MPL-based vaccine since their depletion one day before vaccination resulted in eradication of all tumors (FIG. 4). This finding, demonstrating that MPL efficacy is compromised by Treg cells is significant, and provides an important mechanistic insight for improving therapeutic cancer vaccines.

Although MPL primarily targets cells of innate immunity, a series of recent studies have demonstrated that this adjuvant may also directly target cells of adaptive immunity. The expression of TLR-4 has been shown on CD4⁺ T effector and Treg cells (38, 39). Importantly, stimulation via this receptor on CD4⁺ Teff cells was shown to inhibit ERK1/2 signaling pathway, resulting in the inhibition of their function in an experimental colitis model (39). In marked contrast, stimulation of Treg cells with the TLR-4 agonist lipopolysaccharide resulted in their survival, expansion, and improved regulatory function in vivo (38), which may account for the unfavorable intratumoral CD8⁺ Teff/Treg cell ratio seen in the MPL monotherapy group. Although the exact mechanistic basis of the synergistic effect of SA-4-1BBL and MPL on the intratumoral CD8⁺ T/Treg cell ratio observed in our model is unknown and, without wishing to be bound by theory, the following may apply: (i) SA-4-1BBL may preferentially induce apoptosis in Treg cells as reported for the agonists of OX-40 pathway (40), another close member of TNFR costimulatory family; (ii) SA-4-1BBL may block the tumor-mediated conversion of Teff cells into induced Treg cells and (iii) SA-4-1BBL and MPL may both increase the intratumoral frequency of CD8⁺ Teff cells, thereby favorably influencing the CD8⁺ Teff/Treg cell ratio. These mechanisms are supported by additional (unpublished) data demonstrating that SA-4-1BBL blocks tumor- and TGF-β-induced conversion of Teff cells into induced Treg cells through IFN-γ. The increased expression of IFN-γ in response to an SA-4-1 BBL/MPL adjuvant system is further consistent with this theory. Although enhanced E7 TAA-specific frequency of CD8⁺ T cells expressing IFN-γ was observed in the periphery of mice vaccinated with MPL as monotherapy, this effect did not result in increased number of CD8⁺ T cells in the tumor, suggesting that these cells may not be trafficking into the tumor. In contrast, vaccination with the combined SA-4-1BBL/MPL adjuvant resulted in significantly higher numbers of CD8⁺ Teff cells both in the periphery and within the tumor, suggesting that both adjuvants in combination may affect the trafficking/entry of CD8⁺ Teff into the tumor and/or improve their survival. Thus, and while not wanting to be bound by theory, immunomodulation with SA-4-1BBL is believed to block the conversion of T effector cells into T regulatory cells, which may be a factor for the robust therapeutic efficacy of the MPL/SA-4-1BBL combination.

The therapeutic activity of the combined SA-4-1BBL/MPL adjuvant was achieved in the absence of detectable acute toxicity and chronic autoimmunity. The lack of acute toxicity is consistent with previously published studies demonstrating that treatment of mice with 4-fold higher SA-4-1BBL over the therapeutic dose used in these studies did not result in detectable toxicity, as assessed by systemic cytokine response, non-specific lymphoproliferation, altered lymphocyte trafficking, generalized lymphomegaly and splenomegaly, and hepatitis, all of which were observed with similar doses of an agonistic antibody to 4-1BB receptor (14). The safety of MPL has already been demonstrated both in preclinical and clinical settings (5, 6, 20). Nevertheless, the complete absence of acute or chronic pathologies is surprising in view of the high potency of the adjuvant combination.

The results presented above demonstrate the robust efficacy of the SA-4-1BBL/MPL adjuvant combination for inducing potent CD8⁺ Teff primary and long-term memory responses against TAAs and a favorable intratumoral CD8⁺ Teff/Treg cell ratio that translate into potent therapeutic efficacy in two different tumor models, and which was observed in the absence of detectable acute toxicity or chronic autoimmunity.

In conclusion, the combination of monophosphoryl lipid A (MPL) and 4-1BBL is surprisingly effective as an adjuvant. When administered with an antigen, an MPL/4-1BBL adjuvant combination results in an immune response that is very potent, e.g. curing 100% of cancers in all mice from a single immunization, a result not previously reported. Not only is the response potent, but also of a type that is particularly effective for therapy and for long term immunity. Notably, the MPL/4-1BBL adjuvant combination stimulated the production of CD8+ Teff cells, and resulted in a favorable Teff/Treg ratio. The Teff/Treg ratio is an important predictor of long term survival from cancer. Also, the MPL/4-1BBL adjuvant combination resulted in the infiltration into tumors of CD8+ Teff cells, consistent with an active immune response against the tumor. Despite the strong and effective immune response, the MPL/4-1BBL adjuvant combination composition did not cause any detectable symptoms of acute or chronic toxicity, such as an autoimmune response. Accordingly, a composition comprising MPL and 4-1BBL as an adjuvant is highly effective in inducing an immune response against cancers and tumors, and can also be used against other conditions, such as treatment for, or prophylaxis against, infectious agents. Because MPL is toll-like receptor (TLR) agonist, the present inventor believes that other TLR agonist adjuvants may exhibit a synergistic effect when used with 4-1BBL as described herein.

### REFERENCES

1. Dougan M, Dranoff G. Immune therapy for cancer. Annu Rev Immunol 2009; 27:83-117.
2. Schabowsky R H, Madireddi S, Sharma R, Yolcu E S, Shirwan H. Targeting CD4⁺CD25⁺FoxP3⁺ regulatory T-cells for the augmentation of cancer immunotherapy. Curr Opin Investig Drugs 2007; 8:1002-8.
3. Kawai T, Akira S. The role of pattern-recognition receptors in innate immunity: update on Toll-like receptors. Nat Immunol 2010; 11:373-84.
4. Croft M. The role of TNF superfamily members in T-cell function and diseases. Nat Rev Immunol 2009; 9:271-85.
5. Romanowski B, de Borba P C, Naud P S, et al. Sustained efficacy and immunogenicity of the human papillomavirus (HPV)-16/18 AS04-adjuvanted vaccine: analysis of a randomised placebo-controlled trial up to 6.4 years. Lancet 2009; 374:1975-85.
6. Didierlaurent A M, Morel S, Lockman L, et al. AS04, an aluminum salt- and TLR4 agonist-based adjuvant system, induces a transient localized innate immune response leading to enhanced adaptive immunity. J Immunol 2009; 183:6186-97.
7. Sharma R K, Elpek K G, Yolcu E S, et al. Costimulation as a platform for the development of vaccines: a peptide-based vaccine containing a novel from of 4-1BBL eradicates established tumors. Cancer Res 2009; 69:4319-26.
8. Myers L, Lee S W, Rossi R J, et al. Combined CD137 (4-1BB) and adjuvant therapy generates a developing pool of peptide-specific CD8 memory T cells. Int Immunol 2006; 18:325-33.
9. Lee H W, Nam K O, Park S J, Kwon B S. 4-1BB enhances CD8+ T cell expansion by regulating cell cycle progression through changes in expression of cyclins D and E and cyclin-dependent kinase inhibitor p27kip1. Eur J Immunol 2003; 33:2133-41.
10. Feltkamp M C, Smits H L, Vierboom M P, et al. Vaccination with cytotoxic T lymphocyte epitope-containing peptide protects against a tumor induced by human papillomavirus type 16-transformed cells. Eur J Immunol 1993; 23:2242-9.
11. Lin K Y, Guarnieri F G, Staveley-O'Carroll K F, et al. Treatment of established tumors with a novel vaccine that enhances major histocompatibility class II presentation of tumor antigen. Cancer Res 1996; 56:21-6.
12. Sharma R K, Schabowsky R-H, Srivastava A, et al. 4-1BB ligand as an effective multifunctional immunomodulator and antigen delivery vehicle for the development of therapeutic cancer vaccines. Cancer Res 2010; 70:3945-54.
13. Sharma R K, Yolcu E S, Elpek K G, Shirwan H. Tumor cells engineered to codisplay on their surface 4-1BBL and LIGHT costimulatory proteins as a novel vaccine approach for cancer immunotherapy. Cancer Gene Ther 2010; 17:730-41.
14. Schabowsky R H, Elpek K G, Sharma R K, et al. A novel form of 4-1BBL has better immunomodulatory activity than an agonistic anti-4-1BB Ab without Ab associated severe toxicity. Vaccine 2009; 28:512-22.
15. Bukczynski J, Wen T, Ellefsen K, Gauldie J, Watts T H. Costimulatory ligand 4-1BBL (CD137L) as an efficient adjuvant for human antiviral cytotoxic T cell responses. Proc Natl Acad Sci USA 2004; 101:1291-6.
16. Cannons J L, Lau P, Ghumman B, et al. 4-1BB ligand induces cell division, sustains survival, and enhances effector function of CD4 and CD8 T cells with similar efficacy. J Immunol 2001; 167:1313-24.
17. Watts T H. TNF/TNFR family members in costimulation of T cell responses. Annu Rev Immunol 2005; 23:23-68.
18. Sorenson B S, Banton K L, Frykman N L, Leonard A S, Saltzman D A. Attenuated Salmonella typhimurium with interleukin 2 gene prevents the establishment of pulmonary metastases in a model of osteosarcoma. J Pediatr Surg 2008; 43:1153-8.
19. Cohen P L, Caricchio R, Abraham V, et al. Delayed apoptotic cell clearance and lupus-like autoimmunity in mice lacking the c-mer membrane tyrosine kinase. J Exp Med 2002; 196:135-40.
20. Mata-Haro V, Cekic C, Martin M, et al. The vaccine adjuvant monophosphoryl lipid A as a TRIF-biased agonist of TLR4. Science 2007; 316:1628-32.
21. Curiel T J, Coukos G, Zou L, et al. Specific recruitment of regulatory T cells in ovarian carcinoma fosters immune privilege and predicts reduced survival. Nat Med 2004; 10:942-9.
22. Gobert M, Treilleux I, Bendriss-Vermare N, et al. Regulatory T cells recruited through CCL22/CCR4 are selectively activated in lymphoid infiltrates surrounding primary breast tumors and lead to an adverse clinical outcome. Cancer Res 2009; 69:2000-9.
23. Dannull J, Su Z, Rizzieri D, et al. Enhancement of vaccine-mediated antitumor immunity in cancer patients after depletion of regulatory T cells. J Clin Invest 2005; 115:3623-33.
24. Powell D J, Jr., Felipe-Silva A, Merino M J, et al. Administration of a CD25-directed immunotoxin, LMB-2, to patients with metastatic melanoma induces a selective partial reduction in regulatory T cells in vivo. J Immunol 2007; 179:4919-28.
25. Quezada S A, Peggs K S, Curran M A, Allison J P. CTLA4 blockade and GM-CSF combination immunotherapy alters the intratumor balance of effector and regulatory T cells. J Clin Invest 2006; 116:1935-45.
26. Phan G Q, Yang J C, Sherry R M, et al. Cancer regression and autoimmunity induced by cytotoxic T lymphocyte-associated antigen 4 blockade in patients with metastatic melanoma. Proc Natl Acad Sci USA 2003; 100:8372-7.
27. Burgdorf S, Scholz C, Kautz A, Tampe R, Kurts C. Spatial and mechanistic separation of cross-presentation and endogenous antigen presentation. Nat Immunol 2008; 9:558-66.
28. Wilcox R A, Chapoval A I, Gorski K S, et al. Cutting edge: Expression of functional CD137 receptor by dendritic cells. J Immunol 2002; 168:4262-7.
29. Zhang L, Wang Q, Wang X, et al. Anti-CD137 monoclonal antibody promotes the direct anti-tumor effect mediated by peripheral blood-derived human dendritic cells in vitro. Cell Mol Immunol 2004; 1:71-6.
30. Toka F N, Suvas S, Rouse B T. CD4+ CD25+ T cells regulate vaccine-generated primary and memory CD8+ T-cell responses against herpes simplex virus type 1. J Virol 2004; 78:13082-9.
31. Chen X, Zhou B, Li M, et al. CD4(+)CD25(+)FoxP3(+) regulatory T cells suppress Mycobacterium tuberculosis immunity in patients with active disease. Clin Immunol 2007; 123:50-9.
32. Xu D, Fu J, Jin L, et al. Circulating and liver resident CD4+CD25+ regulatory T cells actively influence the antiviral immune response and disease progression in patients with hepatitis B. J Immunol 2006; 177:739-47.
33. Yu P, Lee Y, Liu W, et al. Intratumor depletion of CD4+ cells unmasks tumor immunogenicity leading to the rejection of late-stage tumors. J Exp Med 2005; 201:779-91.
34. Elpek K G, Lacelle C, Singh N P, Yolcu E S, Shirwan H. CD4⁺CD25⁺ T regulatory cells dominate multiple immune evasion mechanisms in early, but not late phases of tumor development in a B cell lymphoma model. J Immunol 2007; 178:6840-8.
35. Klages K, Mayer C T, Lahl K, et al. Selective depletion of Foxp3+ regulatory T cells improves effective therapeutic vaccination against established melanoma. Cancer Res 2010; 70:7788-99.
36. Teng M W, Ngiow S F, von S B, et al. Conditional regulatory T-cell depletion releases adaptive immunity preventing carcinogenesis and suppressing established tumor growth. Cancer Res 2010; 70:7800-9.
37. Zhou Q, Bucher C, Munger M E, et al. Depletion of endogenous tumor-associated regulatory T cells improves the efficacy of adoptive cytotoxic T-cell immunotherapy in murine acute myeloid leukemia. Blood 2009; 114:3793-802.
38. Caramalho I, Lopes-Carvalho T, Ostler D, et al. Regulatory T cells selectively express toll-like receptors and are activated by lipopolysaccharide. J Exp Med 2003; 197:403-11.
39. Gonzalez-Navajas J M, Fine S, Law J, et al. TLR4 signaling in effector CD4+ T cells regulates TCR activation and experimental colitis in mice. J Clin Invest 2010; 120:570-81.
40. Gough M J, Ruby C E, Redmond W L, et al. OX40 agonist therapy enhances CD8 infiltration and decreases immune suppression in the tumor. Cancer Res 2008; 68:5206-15.
41. Sharma R K, Yolcu E S, Elpek K G, Shirwan H. Tumor cells engineered to codisplay on their surface 4-1BBL and LIGHT costimulatory proteins as a novel vaccine approach for cancer immunotherapy. Cancer Gene Ther 2010; 17:730-41.

1.-23. (canceled)

24. An adjuvant composition comprising 4-1BBL and a toll-like receptor (TLR) agonist.

25. The composition of claim 24, wherein the TLR agonist is a TLR4 agonist.

26. The composition of claim 24, wherein the TLR agonist is lipid A or a chemical analog thereof.

27. The composition of claim 24, wherein the TLR agonist is monophosphoryl lipid A (MPL).

28. The composition of claim 24, wherein the 4-1BBL is comprised in a streptavidin-4-1BBL fusion protein.

29. The composition of claim 24, further comprising OX-40L.

30. The composition of claim 24, further comprising an antigen.

31. The composition of claim 30, wherein the antigen is a cancer or tumor-associated antigen, a self antigen, or an antigen associated with an infectious agent.

32. The composition of claim 28, wherein the antigen is biotinylated and is provided in a conjugate comprising a streptavidin-4-1BBL fusion protein.

33. The composition of claim 28, wherein the 4-1BBL fusion protein forms trimers or higher order structures.

34. The composition of claim 24, further comprising a pharmaceutically-acceptable excipient.

35. The composition of claim 34, wherein the excipient comprises alum.

36. A method of inducing an immune response against an antigen in a subject, comprising administering to the subject (a) the antigen, (b) a TLR agonist and, (c) 4-1BBL.

37. The method of claim 36, wherein the TLR agonist is monophosphoryl lipid A (MPL).

38. The method of claim 36, wherein the 4-1BBL is comprised in a streptavidin-4-1BBL fusion protein.

39. The method of claim 36, wherein the composition further comprises OX-40L.

40. The method of claim 36, further comprising a second administration of (a) the antigen, (b) a TLR agonist, and (c) 4-1BBL.

41. A method of treating a tumor or a cancer in a subject, comprising administering to the subject (a) an antigen associated with the tumor or cancer, (b) a TLR agonist, and (c) 4-1BBL.

42. The method of claim 41, wherein the TLR agonist is monophosphoryl lipid A (MPL).

43. The method of claim 41, wherein the 4-1BBL is comprised in a streptavidin-4-1BBL fusion protein.

44. The method of claim 41, wherein the composition further comprises OX-40L.

45. The method of claim 41, further comprising a second administration of (a) an antigen associated with the tumor or cancer, (b) a TLR agonist, and (c) 4-1BBL.

46. The method of claim 45, wherein the antigen associated with the tumor or cancer is different between the first and second administrations.


SEQUENCE LISTING (5 sequences)

SEQ ID NO: 1 - protein, 9 aa, Human papillomavirus
RAHYNIVTF

SEQ ID NO: 2 - protein, 358 aa, Artificial Sequence
  Description of Artificial Sequence Synthetic polypeptide
MKLCILLAVVAFVGLSLGRSHHHHHHITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSEFRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 3 - protein, 357 aa, Artificial Sequence
  Description of Artificial Sequence Synthetic polypeptide
MKLCILLAVVAFVGLSLGRSHHHHHHITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSEFACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 364 aa, Artificial Sequence
  Description of Artificial Sequence Synthetic polypeptide
RSHHHHHHGGGGSGGGGTITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSAEAAAKEAAAKAAAEFRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 5 - protein, 358 aa, Artificial Sequence
  Description of Artificial Sequence Synthetic polypeptide
RSDYKDDDDKGTITGTWYNQLGSTFIVTAGADGALTGTYESAVGNAESRYVLTGRYDSAPATDGSGTALGWTVAWKNNYRNAHSATTWSGQYVGGAEARINTQWLLTSGATEANAWKSTLVGHDTFTKVKPSAASSAEAAAKEAAAKAAAEFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE